(19) United States
(12) Patent Application Publication
(10) Pub. No.: US 2020/0129240 A1
(43) Pub. Date: Apr. 30, 2020

(54) SYSTEMS AND METHODS FOR INTRAOPERATIVE PLANNING AND PLACEMENT OF IMPLANTS

(71) Applicant: MIRUS LLC, Marietta, GA (US)

(72) Inventors: Angad SINGH, Marietta, GA (US); Daniel BURNHAM, Atlanta, GA (US)

(21) Appl. No.: 16/626,918

(22) PCT Filed: Jul. 2, 2018
(86) PCT No.: PCT/US2018/040596

§ 371 Date: Dec. 27, 2019

Related U.S. Application Data
(60) Provisional application No. 62/527,230, filed on Jun. 30, 2017.
(60) Provisional application No. 62/576,232, filed on Oct. 24, 2017.

(51) Int. Cl. A61B 34/20; A61B 90/00; A61B 34/10; G06T 17/00; G06T 13/40; G06T 7/00

(52) U.S. Cl. CPC A61B 34/20; G06T 19/006; A61B 34/10; G06T 17/00; G06T 13/40; G06T 7/0012; A61B 2090/3945; A61B 2034/2055; A61B 2034/2048; A61B 2034/104; A61B 2034/107; A61B 2090/3937; A61B 2090/3983; G06T 2207/30052; A61B 90/39

(57) ABSTRACT

Systems and methods are provided for estimating relative pose between two or more implants that are rigidly coupled to respective fiducial markers using instrumentation for the purposes of guidance and/or planning. The systems and/or methods can further include receiving visual and sensory information indicative of the relative pose.

### CROSS-REFERENCE TO RELATED APPLICATIONS

This application claims the benefit of U.S. provisional patent application No. 62/527,230, filed on Jun. 30, 2017, and entitled “SYSTEMS AND METHODS FOR COMPUTER ASSISTED PLACEMENT OF IMPLANTS,” and U.S. provisional patent application No. 62/576,232, filed on Oct. 24, 2017, and entitled “SYSTEMS AND METHODS FOR INTRAOPERATIVE PLANNING OF IMPLANT PLACEMENT,” the disclosures of which are expressly incorporated herein by reference in their entireties.

### TECHNICAL FIELD

The present disclosure relates generally to orthopedic surgery including, but not limited to, joints, spine, upper and lower extremities, and maxillofacial surgery and, more particularly, to a system and method for intraoperative planning and placement of implants.

### BACKGROUND

Many orthopedic surgeries, such as those involving the spine, are complex procedures that require a high degree of precision. For example, the spine is in close proximity to delicate anatomical structures such as the spinal cord and nerve roots. In the case of minimally invasive procedures, the problem is compounded by the limited surgical exposure and visibility. Consequently, the risk of misplaced implants or other complications is high.

The surgeon may rely on intraoperative imaging to plan the placement of implants. Alternatively or additionally, the surgeon may rely on intraoperative imaging to guide and assess the placement of implants. However, imaging is typically not real-time; lacks objective/quantitative information; has to be repeated whenever there is movement of the anatomy, surgical instruments, and/or the implants; and exposes the patient and surgical team to harmful radiation over the duration of the procedure.

Some computer/robotically-assisted surgical systems provide a platform for more reliably planning implant placement. Alternatively or additionally, some computer/robotically-assisted surgical systems provide a platform for more reliably estimating implant placement. These systems are typically limited in scope and rely on intra-operative imaging for registration which is time consuming and exposes the surgical team to radiation. Additionally, these systems typically require complex tracking equipment and bulky markers/sensors that limit the ability to track small instruments and anatomy.

The presently disclosed system and associated methods for intra-operative computer-assisted placement and/or assembly of implants are directed to overcoming one or more of the problems set forth above and/or other problems in the art.

### SUMMARY

According to one aspect, the present disclosure is directed to a method for estimating relative pose between at least two implants for real-time intra operative guidance of implant placement. As used herein, relative pose is the three-dimensional (3D) position and/or orientation of one object relative to another object's coordinate frame. The estimated pose is used to update clinically relevant parameters, path trajectories, surgical plan predictions, and/or virtual models for real-time visualization of the surgery. The method also includes estimating a pose of one or more implants. As used herein, pose is defined as the 3D position and/or orientation of an object relative to a reference coordinate frame such as coordinate frame of a camera-based vision system. The relative pose between the implants is estimated by receiving information from a camera-based vision system that tracks one or more fiducial markers coupled to the implants and estimating relative pose between the fiducial markers and their respective implants. The information may optionally be supplemented with information from inertial and/or magnetic sensors. The method may further optionally include registration of the patient's anatomy involving receiving from system information indicative of one or more anatomic reference positions, axes, planes, landmarks, or surfaces.

According to another aspect, the present disclosure is directed to a method for estimating a relative pose between at least two implants for intra operative planning of placement of one or more additional implants relative to the at least two implants. The relative pose is estimated by receiving information from a camera-based vision system that tracks one or more fiducial markers coupled to the implants and estimation of relative pose between fiducial markers and their respective implants. The information may optionally be supplemented with information from inertial and/or magnetic sensors. For example the estimated pose may be used to update clinically relevant plan parameters such as implant shape, implant size, trajectories, surgical plan predictions, and/or models. The method may further optionally include registration of the patient's anatomy involving receiving from system information indicative of one or more anatomic reference positions, axes, planes, landmarks, or surfaces.

In accordance with another aspect, the present disclosure is directed to a system for estimating relative pose between at least two implants. The system includes fiducial markers coupled to the implants. The system also includes one or more imaging devices (e.g., cameras) close to the surgical field, such as mounted on the surgical table or cart with or without an articulating arm with appropriate degrees of freedom and range. Alternatively, the imaging devices may be integrated with surgical lighting or other surgical equipment such as imaging equipment (e.g., X-ray machine or other imaging equipment). Alternatively, the imaging devices may be integrated in a headset such as an Augmented Reality (AR) headset or Virtual Reality (VR) headset that is worn by the surgeon. The system also includes a processor, communicatively coupled to the imaging devices. The processor may be configured to create virtual models of the implants from two-dimensional (2D) or three-dimensional (3D) computer-aided design (CAD) data or images. The processor is also configured to estimate pose of the fiducials and relative pose between fiducial markers and their respective implants which may optionally include performing instrument registration. The processor may further optionally be configured to register one or more axes, planes, landmarks or surfaces associated with a patient's anatomy. The processor may be further configured to estimate the relative pose between the implants during surgery and update plan parameters for placement of one or more additional implants. Alternatively or additionally, the processor may be further configured to estimate the relative pose of the implants in real-time during surgery and animate/visualize the virtual models also in real-time to give the surgeon an accurate visualization of relative pose between the implants. The processor may be further configured to estimate pose between the implants.

The fiducial markers utilized in the system are visual and/or visual-inertial. For example, in some implementations, the fiducial markers are visual fiducial markers. In other implementation, the fiducial markers are combined visual-inertial fiducial markers, meaning inertial sensors are physically coupled to the fiducial marker and/or the implant. Visual refers to features or patterns that are recognizable by a camera or vision system and inertial refers to sensors that measure inertial data such as acceleration, gravity, angular velocity, magnetic fields, etc. For example, the fiducial marker may include an Inertial Measurement Unit and at least one patterned, reflective or light-emitting feature.

In some implementations, the fiducial marker includes planar two dimensional patterns or contoured surfaces. The contoured or patterned surface can aid an imaging system in recognizing the fiducial marker and determine pose of the fiducial marker from the projection of the contoured or patterned feature on the camera image plane. Techniques for determining 3D pose from 2D features on the image plane are well known in the field of computer vision and are available on on-line open source computer vision libraries such as OpenCV (https://opencv.org). Such fiducial markers may be easily attached, etched, or printed on/to any surface such as the surface of an instrument. The pattern may encode information such as a bar code or QR code.

Such information may include a unique identifier as a well as other information to facilitate localization.

Alternatively or additionally, in some implementations, the fiducial marker is a contoured or patterned three dimensional surface.

Alternatively or additionally, in some implementations, the fiducial marker includes a reflective surface. The reflective surface can aid an imaging system in recognizing the fiducial marker and determine pose of the fiducial marker from the projection of the reflective surface on the camera image plane.

Alternatively or additionally, in some implementations, the fiducial marker has one or more light sources. Optionally, the light source can be a light-emitting diode. Alternatively or additionally, the light source can optionally be configured to emit light at a predetermined frequency, which can aid an imaging system in recognizing the fiducial marker and determine pose of the fiducial marker from the projection of the light source on the camera image plane. Alternatively or additionally, the light source can optionally be configured to emit light having a predetermined pattern, which can aid an imaging system in recognizing the fiducial marker.

Alternatively or additionally, in some implementations, the fiducial marker has one or more light/photo sensors. The sensors are configured to detect light from a fixed beacons or reference sources arranged in the proximity of the surgical field.

In some implementations, the fiducial marker can optionally include a diffuser element. The diffuser element can be configured to condition reflected or emitted light. The diffuser element can be a textured glass or polymer housing the contains the entire fiducial marker or be arranged in proximity to or at least partially surrounding the fiducial marker.

In some implementations described herein, the fiducial marker is coupled with an inertial sensor such as an inertial measurement unit including at least one of a gyroscope, an accelerometer, or a magnetometer. Optionally, the inertial measurement unit further includes a network module configured for communication over a network. For example, the network module can be configured for wireless communication.

The image capturing device (sometimes also referred to herein as “imaging device”) utilized in the system may be a visible light monocular or stereo camera (e.g., a red-green-blue (RGB) camera) of appropriate resolution, focal length(s), and/or specific to one or more wavelengths of interest such as infrared. The image capturing device may also be equipped with multi-spectral multi-focal length imaging capabilities to allow simultaneous imaging at different wavelengths and/or focal lengths. The image device may also have one or more illumination sources to illuminate the surgical field and fiducials markers therein with light of one or more wavelengths such as infrared and blue. The image capturing device may be communicatively coupled to the processing unit via a wired connection or wirelessly.

Alternatively or additionally, the image capturing device utilized in the system may be an active depth camera providing depth information in addition to RGB information. Such cameras are commonly referred to as RGB-D cameras. The image capturing device may be communicatively coupled to the processing unit via a wired connection or wirelessly.

An example method for estimating relative pose between two or more implants and updating plan parameters for one or more other implants is described herein. The method can also include receiving, via one or more imaging devices and/or via one or more inertial measurement units, information indicative of the relative pose between the surgical implants. The method also includes estimating relative pose between the fiducial markers and their respective implants which optionally could involve an instrumentation registration process. The method can further optionally include establishing, via a registration process, information indicative of a reference. For example, the reference can include one or more positions, axes, planes, landmarks, or surfaces.

In some implementations, the fiducial marker can optionally include one or more inertial measurement units. Additionally, the method can further include fusing the visual and inertial information wherein the updated pose of the anatomy is estimated based on the fused information. Optionally, the above information is fused using a Kalman filter or an extended Kalman filter.

In some implementations, the method can further include displaying an estimated angle or a position between a plurality of surgical implants and plan parameters for additional implants relative those implants. Non-limited examples of plan parameters are implant shape, size, trajectory, and/or models.

In some implementations, the method can further include displaying an estimated angle and or a position between one or more surgical implants relative to one or more anatomic references and plan parameters for one or more implants relative those implants and/or anatomic references.

Another example method for estimating the relative pose between two or more implants is described herein. The method can include establishing rigid coupling between a plurality of fiducial markers and a plurality of implants, respectively; estimating first information indicative of a respective relative pose between a respective fiducial marker and a respective implant for each fiducial marker-implant set; receiving, via an imaging device, second information indicative of a respective pose of each of the fiducial markers; estimating a respective pose of each of the implants based on the first information and the second information; and estimating a relative pose between at least two implants based on the respective pose of each of the at least two implants.

In some implementations, at least one of the implants is arranged under a patient's skin.

In some implementations, at least one of the implants is not visible to the imaging device.

In some implementations, estimating a respective pose of each of the implants based on the first information and the second information includes, for each fiducial marker-implant set, extrapolating a pose of an implant from a pose of a fiducial marker to which the implant is rigidly coupled.

In some implementations, establishing rigid coupling between a plurality of fiducial markers and a plurality of implants, respectively, includes for each fiducial marker-implant set, rigidly attaching a fiducial marker and an implant at respective specific locations and in specific relative orientations on a surgical instrument.

In some implementations, the first information for each fiducial marker-implant set is estimated from: pre-operative information related to the dimensions of the surgical instrument and the implant; and a respective attachment point and a respective relative orientation of each of the fiducial marker and the implant on the surgical instrument.

In some implementations, the first information is estimated preoperatively and/or intraoperatively via an instrument registration process.

In some implementations, the instrument registration process includes three-dimensional (3D) reconstruction of the implants from one or more images captured by the imaging device. In other implementations, the instrument registration process includes use of a point-pair technique and/or a point-cloud technique.

In some implementations, the method further includes receiving, via an inertial measurement unit, third information indicative of a respective pose of each of the implants.

In some implementations, the method further includes fusing the second information and the third information, wherein the respective pose of each of the implants is estimated based on the first information and the fused second and third information. Optionally, the second information and the third information are fused using a Kalman filter or an extended Kalman filter.

In some implementations, the method further includes tracking the fiducial markers using the imaging device.

In some implementations, at least one of the fiducial markers includes a pattered or contoured surface, a light reflector or a light-emitting source, and/or an inertial measurement unit.

In some implementations, the method further includes displaying an estimated angle or a position between the at least two implants.

In some implementations, the method further includes displaying an estimated angle or a position between at least one of the implants and an anatomic axis or plane.

In some implementations, the method further includes creating a virtual model of at least one of the implants using pre-operative and/or intra-operative information; and displaying a respective pose of the at least one of the implants by animating the virtual model.

In some implementations, the method further includes displaying the animated virtual model using an augmented reality or virtual reality headset.

In some implementations, the method further includes updating a plan parameter using the estimated respective pose of at least one of the implants. The plan parameter is an implant shape, an implant size, a trajectory, a surgical plan prediction, and/or a model of the at least one of the implants.

In some implementations, the method further includes planning placement of the at least one of the implants.

In some implementations, the method further includes displaying the plan parameter.

Another example system for estimating the relative pose between two or more implants is described herein. The system can include an imaging device; a plurality of fiducial markers coupled to a plurality of implants, respectively, using instrumentation; and a processor communicatively coupled to the imaging device. The processor can be configured to estimate first information indicative of a respective relative pose between a respective fiducial marker and a respective implant for each fiducial marker-implant set; receive, via an imaging device, second information indicative of a respective pose of each of the fiducial markers; estimate a respective pose of each of the implants based on the first information and the second information; and estimate a relative pose between at least two implants based on the respective pose of each of the at least two implants.

It should be understood that the above-described subject matter may also be implemented as a computer-controlled apparatus, a computer process, a computing system, or an article of manufacture, such as a computer-readable storage medium.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other. Like reference numerals designate corresponding parts throughout the several views.

FIG. 1 provides a diagrammatic view of an example system used to measure relative pose between surgical implants consistent with certain disclosed embodiments.

FIG. 2 provides a schematic view of example components associated with a system used to measure relative pose between surgical implants, such as that illustrated in FIG. 1.

FIG. 3 is a fiducial marker according to one example described herein.

FIG. 4 is a fiducial marker according to another example described herein.

FIG. 5 is a fiducial marker according to yet another example described herein.

FIG. 6 is a fiducial marker according to yet another example described herein.

FIG. 7 is an example display of plan parameters for a spinal rod implant using the pose measurement of screw heads of spinal pedicle screw implants.

FIG. 8 illustrates an example point-pair technique for instrument registration using a calibrated pointer.

FIG. 9 illustrates an example point-cloud technique for instrument registration using a calibrated pointer.

FIG. 10 illustrates an example technique for instrument registration using 3D reconstruction from one or more images.

FIG. 11 shows example calculations for relative pose between at least two implants.

### DETAILED DESCRIPTION

Unless defined otherwise, all technical and scientific terms used herein have the same meaning as commonly understood by one of ordinary skill in the art. Methods and materials similar or equivalent to those described herein can be used in the practice or testing of the present disclosure. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. The terms “optional” or “optionally” used herein mean that the subsequently described feature, event or circumstance may or may not occur, and that the description includes instances where said feature, event or circumstance occurs and instances where it does not. Ranges may be expressed herein as from “about” one particular value, and/or to “about” another particular value. When such a range is expressed, an aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

Systems and methods consistent with the embodiments disclosed herein are directed to a computer-assisted system to measure the relative pose between two or more implants. In some implementations, this information can be used to guide placement of one or more implants. In other implementations, this information can be used to update plan parameters for placement of one or more implants. As used herein, pose is defined as 3D position (X,Y,Z) and/or orientation (pitch, yaw, roll) with respect to a reference coordinate frame. A reference coordinate frame can include, but is not limited to, the coordinate frame of a camera based imaging system. For example, as described below, it is possible to obtain the pose of an object (e.g., a fiducial marker, an implant, etc.) with respect to a reference coordinate frame such as that of an imaging device. Additionally, as used herein, relative pose is defined as 3D position (X,Y,Z) and/or orientation (pitch, yaw, roll) of one object with respect to another object's coordinate frame. For example, as described below, it is possible to obtain the relative pose (i.e., 3D position and/or orientation) of an implant with respect to the coordinate frame of a fiducial marker. Alternatively or additionally, it is possible to obtain the relative pose (i.e., 3D position and/or orientation) of an implant with respect to the coordinate frame of another implant. Certain exemplary embodiments eliminate or minimize the need for repeated intra-operative imaging (e.g., fluoroscopy, X-ray, or computed tomography (CT)) which can add time and cost to the procedure and subject the patient to unnecessary exposure to potentially harmful radiation.

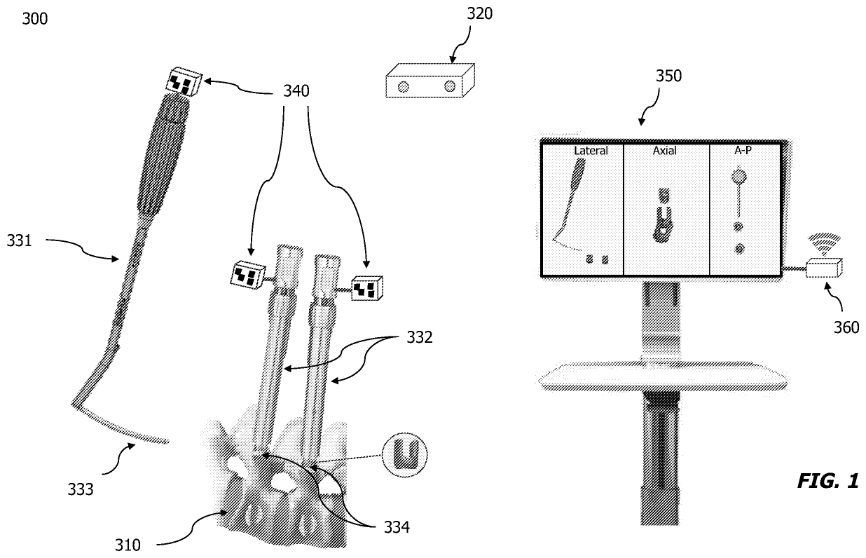

FIG. 1 provides a view depicting an example spine surgical system to measure the relative pose between two or more implants. As illustrated in FIG. 1, the surgical system 300 provides a solution for measuring the relative pose between the implants 333 and 334, and displaying this information in real-time. It should be understood that the spine is only provided as example of the patient's anatomy and that the systems and methods described herein are applicable to anatomy other than the spine. For example, those skilled in the art will recognize that embodiments consistent with the presently disclosed systems and methods may be employed in any environment involving similar surgical procedures.

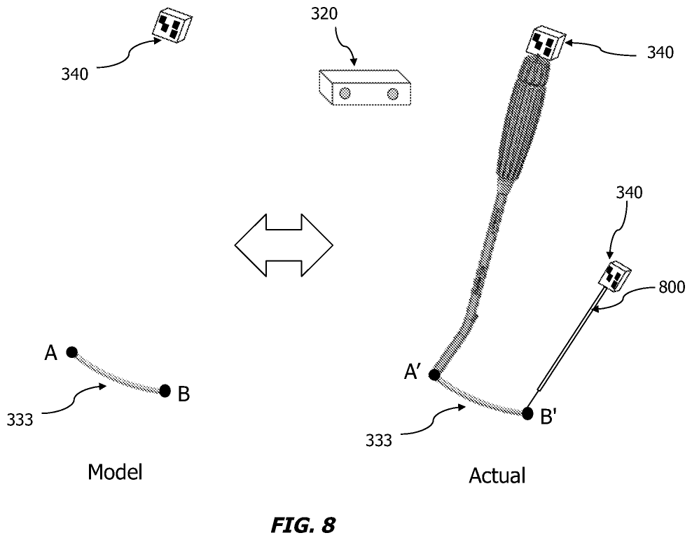

As illustrated in FIG. 1, the system 300 comprises one or more fiducial markers 340, and one or more imaging devices, for example, camera 320. The camera 320 can be operably coupled to a processing and display unit 350. In some embodiments, wireless communication is achieved via wireless communication transceiver 360, which may be operatively connected to processing and display unit 350. Each fiducial marker 340 may contain a feature or features recognizable (e.g., located and identified in the image frame) by the camera 320. In other words, the camera 320 can track the fiducial markers 340 in its field of view (FOV). It should be understood that this provides information indicative of the pose of the fiducial markers 340 (e.g., the “second information” described herein), where the pose of the fiducial markers 340 are relative to the reference coordinate frame of camera 320. Optionally fiducial marker 340 may be coupled to inertial measurement units as described herein. Any number of fiducial markers and inertial measurement units or any combination thereof can be used depending on the specific application and type of information desired. As illustrated in FIG. 1, fiducial markers 340 and implants 333, 334 are rigidly fixed on surgical instruments 331, 332 at specified locations and in specific orientations such that relative pose between respective fiducial markers 340 and implants 333, 334 can be estimated. The relative pose between a fiducial marker (e.g., one or more fiducial markers) and an implant to which it is rigidly coupled is part of the “first information” described herein. In other words, the relative pose represents the 3D position and/or orientation of an implant relative to the coordinate frame of a fiducial marker. For example, in FIG. 1, surgical instrument 331 is a rod insertion surgical instrument to which implant 333 (e.g., a rod) and fiducial marker 340 are rigidly attached. Surgical instrument 331 (e.g., a rod insertion surgical instrument) is used for inserting implant 333 (e.g., a rod) through implants 334 (e.g., tulip heads). Implants 334 (e.g., screw heads) and respective fiducial marker 340 are rigidly attached to surgical instruments 332. It should be understood that implants 334 (e.g., screw heads) are arranged under the patient's skin (e.g., subcutaneous) such that implants 334 are not visible. Such coupling between the fiducial markers and implants using instrumentation allows for tracking of the implant pose even when the implant is under the skin (i.e., not visible to camera 320) as is the case for minimally invasive procedures. An example method to determine the relative pose between the fiducial marker 340 and the surgical instrument 331,332 and/or implant 333, 334 is to utilize known dimensions (either from CAD models or actual measurements) and known rigid arrangement of connected fiducials, instruments, and implants. Alternatively, the system may determine the relative pose between the fiducial marker 340 and the surgical instrument 331, 332 and/or implant 333,334 via an instrument registration process as described below with regard to FIGS. 8-10. Such a process may take into account and compensate for any deviations due to manufacturing/assembly variations and/or any changes that have made to the implant by the surgeon prior to insertion, such as changes made as result implant planning as described later. For example if a spinal rod implant 333 is bent, cut or otherwise reshaped in the planning process or otherwise, the instrument registration process can be utilized to determine a new corrected relative pose between the fiducial marker 340, the surgical instrument 331, and implant 333 as well as update any virtual models of implant 333 for visualization on processing and display unit 350.

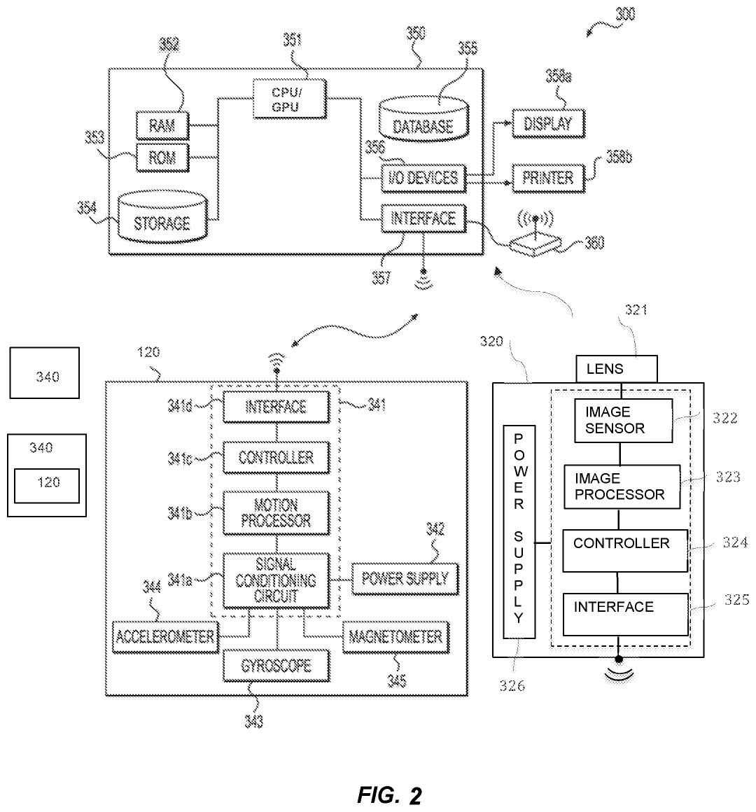

Referring now to FIG. 8, an example point-pair method for instrument registration using a calibrated pointer is shown. The goal of the instrument registration is to make relative pose between fiducial marker 340 and implant 333 in the model (i.e., shown by left-hand side of FIG. 8) match the actual rigid assembly of fiducial marker 340 and implant 333 in use (i.e., shown by right-hand side of FIG. 8). First, the example method shown in FIG. 8 relies on a calibrated pointer 800 including a respective fiducial marker 340. The pointer 800 is calibrated such that position of the tip of pointer 800 from the respective fiducial marker 340 is known with a high degree of precision. As the pose of fiducial marker 340 is tracked by camera 320, the position of the tip of pointer 800 is therefore also tracked. Pointer 800 is used to touch two or more specific points, shown as points A′ and B′ on the actual implant 333, corresponding to points A and B on the model. These points and pose of fiducial marker 340 coupled implant 333 are collected and stored by a processing unit (e.g., processing and display unit 350 of FIGS. 1 and 2). Using point-pair registration techniques known in the art, the model can then be registered/matched to the actual instrument.

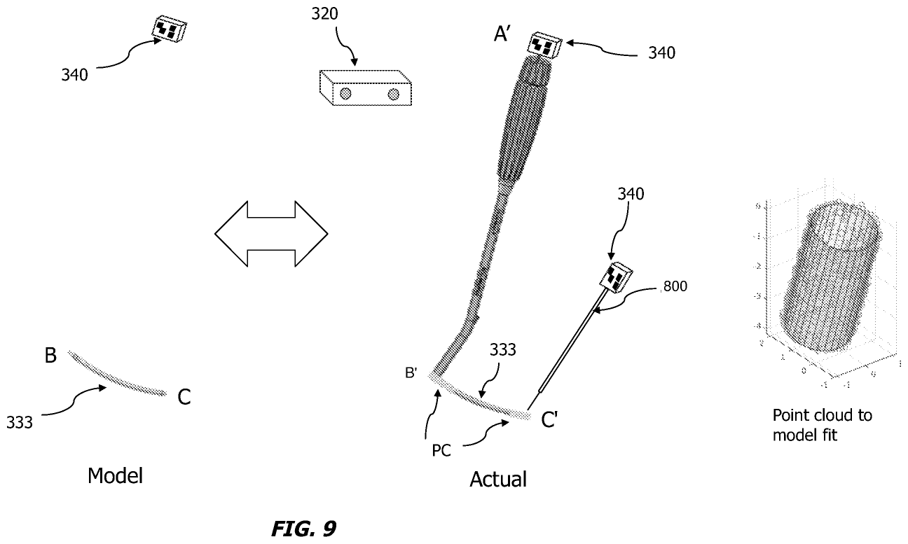

Referring now to FIG. 9, an example point-cloud method for instrument registration using a calibrated pointer is shown. In this method, calibrated pointer 800 as described above with regard to FIG. 8 is used to trace one or more specific features or regions on implant 333. Similarly to FIG. 8, the goal of the instrument registration is to make relative pose between fiducial marker 340 and implant 333 in the model (i.e., shown by left-hand side of FIG. 9) match the actual rigid assembly of fiducial marker 340 and implant 333 in use (i.e., shown by right-hand side of FIG. 9). The positions of points touched/traced by the pointer 800 (i.e. point cloud ‘PC’) and pose of fiducial marker 340 coupled to implant 333 are collected and stored by a processing unit (e.g., processing and display unit 350 of FIGS. 1 and 2). These points or point clouds are then registered to the model using algorithms known in the art. Examples of algorithms to perform the above registration are Iterative Closest Point (and its many variants), Principal Component Analysis, and Singular Value Decomposition. It should be understood that the above algorithms are provided only as examples and that other registration algorithms can be used.

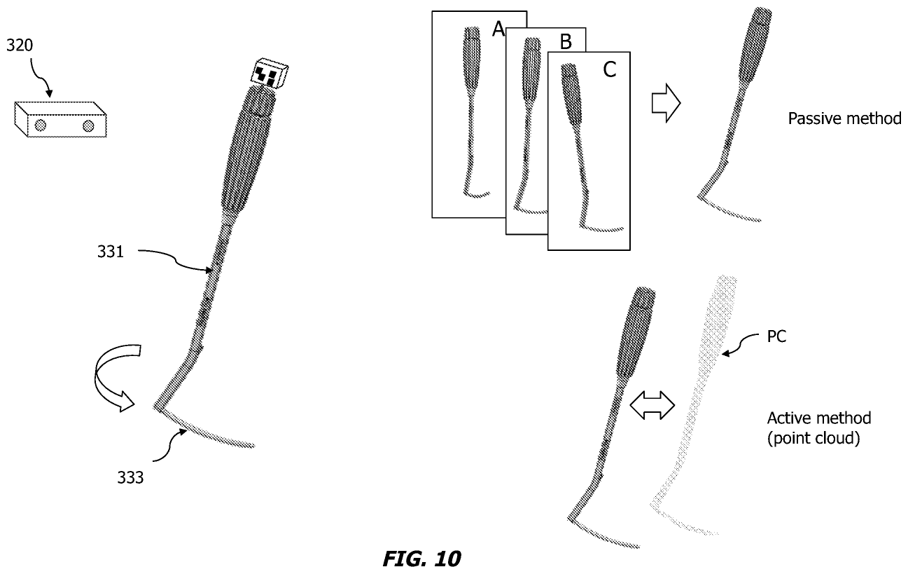

Referring now to FIG. 10, an example method for instrumentation registration using 3D reconstruction from one or more images is shown. This disclosure contemplates that the image(s) can be captured using camera 320. In computer vision, the process of capturing the 3D shape and appearance of real objects is broadly termed “3D reconstruction” and two methods, one passive and one active are described herein. The passive method utilizes 2D images captured by a calibrated digital camera 320. Typically, two or more images at different view angles are needed for the passive method. As shown in FIG. 10, images A, B, and C of the instrument 331 with fiducial marker 340 attached thereto are collected and stored by a processing unit (e.g., processing and display unit 350 of FIGS. 1 and 2). Algorithms to extract features and establish correspondences are then performed by processing unit. It should be understood that such algorithms for extracting features and establishing correspondences are known in the art. Finally, 3D pose information is recovered and registered to the model. In the active method, also shown in FIG. 10, camera 320 is a 3D camera such as a depth camera (e.g. structured light, time of flight, laser rangefinder/scanner, etc.). Such cameras generate point clouds/depth images of objects in its field of view. For increased accuracy and completeness of the data, multiple depth images of the instrument assembly at different view angles may be collected by moving the instrument 331 or the camera 320. These multiple perspectives can then be stitched together using 3D reconstruction techniques know in the art. The data is then registered to the model using techniques known in the art. Examples of algorithms to perform the above registration are Iterative Closest Point (and its many variants), Principal Component Analysis, and Singular Value Decomposition. It should be understood that the above algorithms are provided only as examples and that other registration algorithms can be used. In both the passive and active methods above, the 3D reconstructed data may optionally be segmented prior to registration using color and/or depth information and/or by defining a volume in close proximity to fiducial marker 340 as a “region of interest”.

Optionally, along with the instrument registration methods described above, a deformable model may be utilized, wherein the model may be programmatically/virtually deformed along certain axes or planes to account for any dimensional or shape changes made to the implant by the surgeon or otherwise.

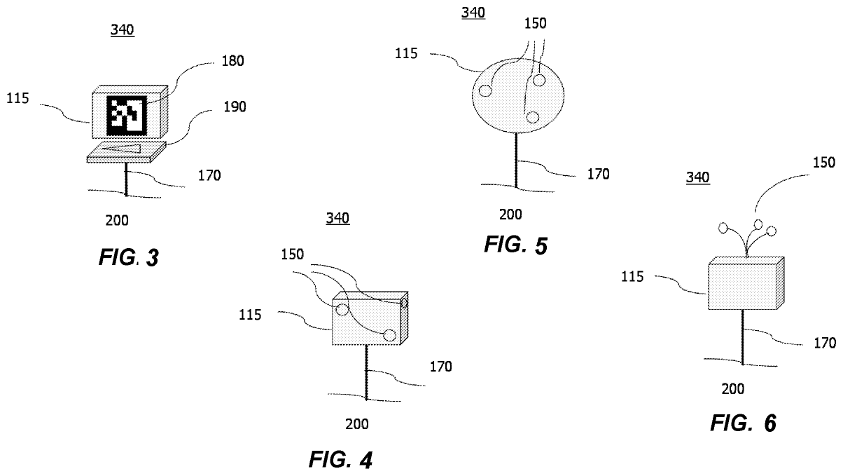

Referring now to FIGS. 3-6 example fiducial markers 340 according to implementations described herein are shown. In a general sense in the field of computer vision, a fiducial marker is a known object in the camera field of view (FOV) that can be recognized in each image frame. Therefore, there are numerous examples of two-dimensional (2D) (e.g., planar) and three-dimensional (3D) fiducial markers well known in the field and suitable for use in the system as shown in FIG. 1. FIGS. 3-6 are a few representative examples and should not be construed as limiting the disclosure in any way. Fiducial marker 340 as envisioned in the disclosed system can either be a purely visual marker containing visual features for localization, identification, and tracking by the camera-based vision system. Alternatively, fiducial marker 340 can optionally include inertial measurement units in addition to the visual features. An example inertial measurement unit is described below. As described below, the inertial measurement unit can be incorporated into the housing 115 of fiducial marker 340 or rigidly coupled to 340 via other suitable means such as adhesives or mechanical constructs.

In one embodiment, fiducial marker 340 contains a 2D or 3D patterned surface 180 (e.g., a checkered pattern, dot pattern, or other pattern) as shown in FIG. 3. The pattern can optionally be distinctive or conspicuous such that the patterned surface contains multiple detectable features that aid an imaging system in recognizing the fiducial marker 340. The pattern can also encode a distinctive identifier and/or digital payload similar to a Quick Response (QR) code. Alternatively, the fiducial marker 340 contains a 2D or 3D contoured surface. The contoured surface can optionally be distinctive or conspicuous such that the surface contains multiple features that aid an imaging system in recognizing the fiducial marker 340. The patterned or contoured surface as described above may optionally be etched or printed onto the surface of instruments 332 and 331.

In another embodiment, fiducial marker 340 can include of a reflective or light-emitting source 150 (referred to herein as “source(s) 150”). For example, each of the fiducial markers 340 of FIGS. 3-6 includes a plurality of sources 150 (e.g., 3 sources). It should be understood that FIGS. 3-6 are provided only as examples and that the fiducial marker 340 can include any number of sources 150. In addition, the sources 150 can be arranged in a fixed pose with respect to one another. The fixed pose can be distinctive or conspicuous such that the fiducial marker 340 can be recognized by the imaging system. The source 150 can be made of reflective material such that the source 150 reflects incident light. Alternatively or additionally, the source 150 can be a light source, e.g., a light-emitting diode or other light source. Additionally, the light source can optionally be configured to emit light at a predetermined frequency. Alternatively or additionally, the light source can optionally be configured to emit light having a predetermined pattern. It should be understood that providing emitted light with a predetermined frequency and/or pattern can aid an imaging system in recognizing and/or uniquely identifying the fiducial marker 340.

The fiducial marker 340 can include a housing 115. The housing 115 can enclose one or more components (described below) of the fiducial marker 340. Optionally, the source 150 can be integrated with the housing. For example, the source 150 can be integrated with an outer (e.g., exterior) surface of the housing 115 as shown in FIGS. 3-6. Alternatively or additionally, the source 150 can optionally be attached to or extend from the housing 115. For example, the source 150 can be attached to or extend from the outer surface of the housing 115 as shown in FIG. 6. Optionally, the housing 115 can define a patterned surface (e.g., a checkered pattern or other pattern) as discussed above with regard to FIG. 3. For example, at least a portion of the outer surface of the housing 115 can contain the pattern. Optionally, the housing 115 can include a contoured surface. For example, at least a portion of the outer surface of the housing 115 can be contoured. The contoured surface can optionally be distinctive or conspicuous such that the surface contains multiple detectable features that aid an imaging system in recognizing the fiducial marker 340. It should be understood that the fiducial marker 340 shown in FIGS. 3-6 are provided only as examples and that the fiducial marker and/or its housing can be other shapes and/or sizes.

The fiducial marker 340 can include a quick connect feature such as a magnetic quick connect to allow for easy fixation to a base plate such as, for example, a base plate 190 shown in FIG. 3. The mating surface of the fiducial 340 and the base plate 190 may have a suitable keyed feature that ensure fixation of fiducial 340 to the base plate 190 in a fixed orientation and position.

The fiducial marker 340 or base plate 190 (if present) can include an elongate pin 170 as shown in FIG. 3-6. Alternatively or additionally, the elongate pin 170 can optionally have a tapered distal end. Alternatively or additionally, the elongate pin 170 can optionally have a threaded distal end. The distal end can be configured to anchor the fiducial marker 340 to another object 200 such as a surgical instrument, for example.

Alternatively fiducial marker 340 can have a threaded ends (surfaces) that screw on to the instruments.

Optionally, the fiducial marker 340 can include a diffuser element. The diffuser element can be configured to condition reflected or emitted light. For example, the diffuser element can be configured to diffuse or scatter reflected or emitted light. Such light can be from an illumination source on camera 320. Optionally, the diffuser element can be a textured glass or polymer housing for enclosing or containing the source 150. The diffuser element can optionally be arranged in proximity to or at least partially surrounding the fiducial. Alternatively or additionally, the fiducial marker 340 can optionally include at least one of a magnetic field generator or an acoustic transducer. Alternatively or additionally, the fiducial marker 340 can include a photosensor (e.g., a light measuring device) such as a photodiode, for example, configured to detect light from one or more illumination sources such as on camera 320. The illumination sources may optionally be configured to flash and/or strobe the light at specific frequencies/time intervals and/or specific patterns.

Note that although there is no technical limitation on the number of fiducial markers that can be used, a practical limit is expected to be around 100 fiducials. However, the quantity of fiducial markers used does not interfere with or limit the disclosure in any way. As noted above, it should be understood that the surgical instruments and/or implants for spinal surgery are provided only as examples and that the systems and/or method for computer-assisted implant placement can be used with surgical procedures on different parts of the anatomy.

As discussed herein, the fiducial marker 340 can optionally include inertial measurement units. In this case, the housing 115 of the fiducial marker 340 can enclose one or more components (described below) of an inertial measurement unit. Depending on the embodiment of fiducial marker 340 as previously discussed, the respective visual features may be integrated within or on the housing 115. For example, a 2D or 3D patterned surface can be integrated with an outer (e.g., exterior) surface of the housing 115 as shown in FIG. 3. For example, the source 150 can be integrated with an outer (e.g., exterior) surface of the housing 115 as shown in FIGS. 3 and 4. Alternatively or additionally, the source 150 can optionally be attached to or extend from the housing 115 as shown in FIG. 6. It should be understood that FIGS. 3-6 are provided only as examples and that the housing 115 of fiducial marker 340 containing the inertial measurement unit can be in other shapes and/or sizes.

In certain embodiments, it may be necessary to harmonize the respective coordinate frames of the fiducial marker (e.g., fiducial marker 340) and inertial measurement unit (e.g., inertial measurement unit 120) via a calibration procedure that determines the fixed transform between the respective coordinate frames. Such calibration procedures are well-known in the art. For example, such a procedure could consist of collecting data as the visual-inertial fiducials are moved in a pattern and then iteratively solving the data for the fixed relative transform between the two coordinate frames.

Inertial measurement unit may include one or more subcomponents configured to detect and transmit information that either represents the pose or can be used to derive the pose of any object that is affixed relative to inertial measurement unit, such as a surgical instrument.

Inertial measurement unit 120 consistent with the disclosed embodiments is described in greater detail below with respect to the schematic diagram of FIG. 2. According to one embodiment, as shown in FIG. 2 may include or embody one or more of gyroscopes and accelerometers. The inertial measurement unit 120 may also include magnetic sensors such as magnetometers. Inertial measurement units measure earth's gravity as well as linear and rotational motion that can be processed to calculate pose relative to a reference coordinate frame. Magnetic sensors measure the strength and/or direction of a magnetic field, for example the strength and direction of the earth's magnetic field or a magnetic field emanating from magnetic field generator. Using “sensor fusion” algorithms, some of which are well known in the art, the inertial measurement units and/or magnetic sensors may combine to measure full 3/6 degree-of-freedom (DOF) motion and pose relative to a reference coordinate frame such as a global reference frame defined by North-East-Down.

Inertial measurement unit 120 associated with the presently disclosed system may each be configured to communicate wirelessly with each other and to a processing and display unit 350 that can be a laptop computer, PDA, or any portable, wearable (such as augmented/virtual reality glasses or headsets) or desktop computing device. The wireless communication can be achieved via any standard radio frequency communication protocol such Bluetooth, Wi Fi, ZigBee, etc., or a custom protocol. In some embodiments, wireless communication is achieved via wireless communication transceiver 360, which may be operatively connected to processing and display unit 350.

The processing and display unit 350 runs software that calculates the pose of the surgical instruments 331, 332 and implants 333, 334 based on the visual and/or inertial measurement unit information and displays the information on a screen in a variety of ways based on surgeon preferences including overlaying of virtual information on real anatomic views as seen by the surgeon so as to create an augmented reality view. For example such visualization can be provided using 2D/3D augmented/mixed reality headsets that are becoming increasing common. Examples of such headsets are the Hololens from Microsoft, Meta 2 from the Meta Company, and Magic Leap One from Magic Leap. The surgeon or surgical assistants can interact with the processing unit either via a keyboard, wired or wireless buttons, touch screens, voice activated commands, or any other technologies that currently exist or may be developed in the future.

In addition to their role as described above, fiducial marker 340 and/or inertial measurement units 120 also allow a means for the system to register instruments and/or anatomic axes, planes, surfaces, and/or features as described herein. As described herein, in some implementations, the fiducial marker 340 is purely a visual fiducial marker. Alternatively or additionally, in other implementations, the fiducial marker 340 can incorporate an inertial measurement unit 120. Optionally, inertial measurement unit 120 can be used for registration alone.

FIG. 2 provides a schematic diagram illustrating certain exemplary subsystems associated with system 300 and its constituent components. Specifically, FIG. 2 is a schematic block diagram depicting exemplary subcomponents of processing and display unit 350, fiducial marker 340, inertial measurement unit 120, and imaging device such as a camera 320. As described herein, this disclosure contemplates that the camera can be a monocular or stereo digital camera (e.g., RGB camera), and/or active depth camera (e.g. structured light, time of flight, laser rangefinder/scanner), an infrared camera, and/or a multi-spectral multi-focal length imaging camera or some combination of the above. Alternatively, the camera can be a system of individual cameras placed around the surgical field.

For example, in accordance with the exemplary embodiment illustrated in FIG. 2, system 300 may embody a system for intra-operatively—and in real-time or near real-time—measuring relative pose between two or more implants. As illustrated in FIG. 2, system 300 may include a processing device (such as processing and display unit 350 (or other computer device for processing data received by system 300)), and one or more wireless communication transceivers 360 for communicating with the sensors attached to the patient's anatomy (not shown). The components of system 300 described above are examples only, and are not intended to be limiting. Indeed, it is contemplated that additional and/or different components may be included as part of system 300 without departing from the scope of the present disclosure. For example, although wireless communication transceiver 360 is illustrated as being a standalone device, it may be integrated within one or more other components, such as processing and display unit 350. Thus, the configuration and arrangement of components of system 300 illustrated in FIG. 2 are intended to be examples only.

Processing and display unit 350 may include or embody any suitable microprocessor-based device configured to process and/or analyze information indicative of the pose of an anatomy and/or surgical instrument. According to one embodiment, processing and display unit 350 may be a general purpose computer programmed with software for receiving, processing, and displaying information indicative of the pose of the anatomy and/or surgical instrument. According to other embodiments, processing and display unit 350 may be a special-purpose computer, specifically designed to communicate with, and process information for, other components associated with system 300. Individual components of, and processes/methods performed by, processing and display unit 350 will be discussed in more detail below.

Processing and display unit 350 may be communicatively coupled to the fiducial marker(s) 340, the inertial measurement unit(s) 120, and camera(s) 320 and may be configured to receive, process, and/or analyze sensory and/or visual data measured by the fiducial marker 340 and/or camera 320. Processing and display unit 350 may also be configured to receive, process, and/or analyze sensory data measured by the inertial measurement unit(s) 120. According to one embodiment, processing and display unit 350 may be wirelessly coupled to fiducial marker(s) 340, the inertial measurement unit(s) 120, and camera(s) 320 via wireless communication transceiver(s) 360 operating any suitable protocol for supporting wireless (e.g., wireless USB, ZigBee, Bluetooth, Wi-Fi, etc.) In accordance with another embodiment, processing and display unit 350 may be wirelessly coupled to fiducial marker(s) 340, the inertial measurement unit(s) 120, and camera(s) 320, which, in turn, may be configured to collect data from the other constituent sensors and deliver it to processing and display unit 350. In accordance with yet another embodiment, certain components of processing and display unit 350 (e.g. I/O devices 356) may be suitably miniaturized for integration using available technologies such as embedded processors and/or Field Programmable Gate Arrays (FPGA) with fiducial marker(s) 340, the inertial measurement unit(s) 120, and camera(s) 320.

Wireless communication transceiver(s) 360 may include any device suitable for supporting wireless communication between one or more components of system 300. As explained above, wireless communication transceiver(s) 360 may be configured for operation according to any number of suitable protocols for supporting wireless, such as, for example, wireless USB, ZigBee, Bluetooth, Wi-Fi, or any other suitable wireless communication protocol or standard. According to one embodiment, wireless communication transceiver 360 may embody a standalone communication module, separate from processing and display unit 350. As such, wireless communication transceiver 360 may be electrically coupled to processing and display unit 350 via USB or other data communication link and configured to deliver data received therein to processing and display unit 350 for further processing/analysis. According to other embodiments, wireless communication transceiver 360 may embody an integrated wireless transceiver chipset, such as the Bluetooth, Wi-Fi, NFC, or 802.11x wireless chipset included as part of processing and display unit 350.

As explained, processing and display unit 350 may be any processor-based computing system that is configured to receive pose information associated with an anatomy or surgical instrument, store anatomic registration information, analyze the received information to extract data indicative of the pose of the surgical instrumentation with respect to the patient's anatomy, and output the extracted data in real-time or near real-time. Non-limiting examples of processing and display unit 350 include a desktop or notebook computer, a tablet device, a smartphone, wearable computers including augmented/virtual reality glasses or headsets, handheld computers, or any other suitable customized or off-the-shelf processor-based computing system.

For example, as illustrated in FIG. 2, processing and display unit 350 may include one or more hardware and/or software components configured to execute software programs, such as algorithms for tracking pose of objects such as the surgical implants. This disclosure contemplates using any algorithm known in the art for tracking such pose. According to one embodiment, processing and display unit 350 may include one or more hardware components such as, for example, a central processing unit (CPU), Graphics processing unit (GPU), or microprocessor 351, a random access memory (RAM) module 352, a read-only memory (ROM) module 353, a memory or data storage module 354, a database 355, one or more input/output (I/O) devices 356, and an interface 357. Alternatively and/or additionally, processing and display unit 350 may include one or more software media components such as, for example, a computer-readable medium including computer-executable instructions for performing methods consistent with certain disclosed embodiments. It is contemplated that one or more of the hardware components listed above may be implemented using software. For example, storage 354 may include a software partition associated with one or more other hardware components of processing and display unit 350. Processing and display unit 350 may include additional, fewer, and/or different components than those listed above. It is understood that the components listed above are examples only and not intended to be limiting.

CPU/GPU 351 may include one or more processors, each configured to execute instructions and process data to perform one or more functions associated with processing and display unit 350. As illustrated in FIG. 2, CPU/GPU 351 may be communicatively coupled to RAM 352, ROM 353, storage 354, database 355, I/O devices 356, and interface 357. CPU/GPU 351 may be configured to execute sequences of computer program instructions to perform various processes, which will be described in detail below. The computer program instructions may be loaded into RAM 352 for execution by CPU/GPU 351.

RAM 352 and ROM 353 may each include one or more devices for storing information associated with an operation of processing and display unit 350 and/or CPU/GPU 351. For example, ROM 353 may include a memory device configured to access and store information associated with processing and display unit 350, including information for identifying, initializing, and monitoring the operation of one or more components and subsystems of processing and display unit 350. RAM 352 may include a memory device for storing data associated with one or more operations of CPU/GPU 351. For example, ROM 353 may load instructions into RAM 352 for execution by CPU/GPU 351.

Storage 354 may include any type of mass storage device configured to store information that CPU/GPU 351 may need to perform processes consistent with the disclosed embodiments. For example, storage 354 may include one or more magnetic and/or optical disk devices, such as hard drives, CD-ROMs, DVD-ROMs, or any other type of mass media device. Alternatively or additionally, storage 354 may include flash memory mass media storage or other semiconductor-based storage medium.

Database 355 may include one or more software and/or hardware components that cooperate to store, organize, sort, filter, and/or arrange data used by processing and display unit 350 and/or CPU/GPU 351. For example, database 355 may include historical data such as, for example, stored placement, pose, camera images, and point cloud data associated with surgical procedures. CPU/GPU 351 may access the information stored in database 355 to provide a comparison between previous surgeries and the current (i.e., real-time) surgery. CPU/GPU 351 may also analyze current and previous surgical parameters to identify trends in historical data. These trends may then be recorded and analyzed to allow the surgeon or other medical professional to compare the pose parameters with different prosthesis designs and patient demographics. It is contemplated that database 355 may store additional and/or different information than that listed above. It is also contemplated that the database could reside on the “cloud” and be accessed via an internet connection using interface 357.

I/O devices 356 may include one or more components configured to communicate information with a user associated with system 300. For example, I/O devices may include a console with an integrated keyboard and mouse to allow a user to input parameters associated with processing and display unit 350. I/O devices 356 may also include a display including a graphical user interface (GUI) for outputting information on a display monitor 358a. In certain embodiments, the I/O devices may be suitably miniaturized and integrated with fiducial marker 340, the inertial measurement unit(s) 120, or camera 320. I/O devices 356 may also include peripheral devices such as, for example, a printer 358b for printing information associated with processing and display unit 350, a user-accessible disk drive (e.g., a USB port, a floppy, CD-ROM, or DVD-ROM drive, etc.) to allow a user to input data stored on a portable media device, a microphone, a speaker system, or any other suitable type of interface device.

Interface 357 may include one or more components configured to transmit and receive data via a communication network, such as the Internet, a local area network, a workstation peer-to-peer network, a direct link network, a wireless network, or any other suitable communication platform. For example, interface 357 may include one or more modulators, demodulators, multiplexers, demultiplexers, network communication devices, wireless devices, antennas, modems, and any other type of device configured to enable data communication via a communication network. According to one embodiment, interface 357 may be coupled to or include wireless communication devices, such as a module or modules configured to transmit information wirelessly using Wi-Fi, Bluetooth, or cellular wireless protocols. Alternatively or additionally, interface 357 may be configured for coupling to one or more peripheral communication devices, such as wireless communication transceiver 360.

According to one embodiment, inertial measurement unit 120 may be an integrated unit including a microprocessor 341, a power supply 342, and one or more of a gyroscope 343, an accelerometer 344, or a magnetometer 345. According to one embodiment, inertial measurement unit may contain a 3-axis gyroscope 343, a 3-axis accelerometer 344, and a 3-axes magnetometer 345. It is contemplated, however, that fewer of these devices with fewer axes can be used without departing from the scope of the present disclosure. For example, according to one embodiment, inertial measurement unit 120 may include only a gyroscope and an accelerometer, the gyroscope for calculating the orientation based on the rate of rotation of the device, and the accelerometer for measuring earth's gravity and linear motion or lack of motion (i.e. no motion states). The accelerometer may provide corrections to the rate of rotation information (based on errors introduced into the gyroscope because of device movements that are not rotational or errors due to biases and drifts). In other words, the accelerometer may be used to correct the orientation information collected by the gyroscope. Similarly, the magnetometer 345 can be utilized to measure a magnetic field and can be utilized to further correct gyroscope errors and also correct accelerometer errors. The use of redundant and complementary devices increases the resolution and accuracy of the pose information. The data streams from multiple sensors may be “fused” using appropriate sensor fusion and filtering techniques. An example of a technique that may be suitable for use with the systems and methods described herein is a Kalman Filter or Extended Kalman filter.

As illustrated in FIG. 2, microprocessor 341 of inertial measurement unit 120 may include different processing modules or cores, which may cooperate to perform various processing functions. For example, microprocessor 341 may include, among other things, an interface 341d, a controller 341c, a motion processor 341b, and signal conditioning circuitry 341a. Controller 341c may also be configured to control and receive conditioned and processed data from one or more of gyroscope 343, accelerometer 344, and magnetometer 345 and transmit the received data to one or more remote receivers. The data may be pre-conditioned via signal conditioning circuitry 341a, which includes amplifiers and analog-to-digital converters or any such circuits. The signals may be further processed by a motion processor 341b. Motion processor 341b may be programmed with “sensor fusion” algorithms as previously discussed (e.g., Kalman filter or extended Kalman filter) to collect and process data from different sensors to generate error corrected pose information. The orientation component of the pose information may be a mathematically represented as an orientation or rotation quaternion, euler angles, direction cosine matrix, rotation matrix of any such mathematical construct for representing orientation known in the art. Accordingly, controller 341c may be communicatively coupled (e.g., wirelessly via interface 341d as shown in FIG. 2, or using a wireline protocol) to, for example, processing and display unit 350 and may be configured to transmit the pose data received from one or more of gyroscope 343, accelerometer 344, and magnetometer 345 to processing and display unit 350, for further analysis.

Interface 341d may include one or more components configured to transmit and receive data via a communication network, such as the Internet, a local area network, a workstation peer-to-peer network, a direct link network, a wireless network, or any other suitable communication platform. For example, interface 341d may include one or more modulators, demodulators, multiplexers, demultiplexers, network communication devices, wireless devices, antennas, modems, and any other type of device configured to enable data communication via a communication network. According to one embodiment, interface 341d may be coupled to or include wireless communication devices, such as a module or modules configured to transmit information wirelessly using Wi-Fi or Bluetooth wireless protocols. As illustrated in FIG. 2, inertial measurement unit 120 may be powered by power supply 342, such as a battery, fuel cell, MEMs micro-generator, or any other suitable compact power supply.

Importantly, although microprocessor 341 of inertial measurement unit 120 is illustrated as containing a number of discrete modules, it is contemplated that such a configuration should not be construed as limiting. Indeed, microprocessor 341 may include additional, fewer, and/or different modules than those described above with respect to FIG. 2, without departing from the scope of the present disclosure. Furthermore, in other instances of the present disclosure that describe a microprocessor are contemplated as being capable of performing many of the same functions as microprocessor 341 of inertial measurement unit 120 (e.g., signal conditioning, wireless communications, etc.) even though such processes are not explicitly described with respect to microprocessor 341. Those skilled in the art will recognize that many microprocessors include additional functionality (e.g., digital signal processing functions, data encryption functions, etc.) that are not explicitly described here. Such lack of explicit disclosure should not be construed as limiting. To the contrary, it will be readily apparent to those skilled in the art that such functionality is inherent to processing functions of many modern microprocessors, including the ones described herein.

Microprocessor 341 may be configured to receive data from one or more of gyroscope 343, accelerometer 344, and magnetometer 345, and transmit the received data to one or more remote receivers. Accordingly, microprocessor 341 may be communicatively coupled (e.g., wirelessly (as shown in FIG. 2, or using a wireline protocol) to, for example, processing and display unit 350 and configured to transmit the orientation and position data received from one or more of gyroscope 343, accelerometer 344, and magnetometer 345 to processing and display unit 350, for further analysis. As illustrated in FIG. 2, microprocessor 341 may be powered by power supply 342, such as a battery, fuel cell, MEMs micro-generator, or any other suitable compact power supply.

As shown in FIG. 1 system 300 may further comprise a vision system consisting of one or more cameras 320 that are communicatively coupled, either wirelessly or using a wireline protocol, to display unit 350 and be controlled by CPU/GPU 351. Camera 320 may be placed anywhere in close proximity to the surgery as along as fiducial markers of interest can be clearly imaged. For example, the camera 320 may be rigidly attached to the surgical tables or carts using clamps, bolts, or other suitable means. In yet another embodiment, as shown in FIG. 2, camera 320 may be integrated with overhead surgical lighting or any other appropriate equipment in the operating room such as IV poles, X-ray or other imaging equipment. In yet another embodiment, the imaging devices may be integrated in a headset such as a Augmented Reality or Virtual Reality headset that is worn by the surgeon.

This disclosure contemplates that any commercially available high definition (HD) digital video cameras or any combination thereof such as the Panasonic HX-A1 of Panasonic corp. of Kadoma, Japan can be used. In some embodiments, cameras of different focal lengths may be combined to optimized field of view and accuracy. As shown in FIG. 2, camera 320 may comprise components that are commonly found in digital cameras. For example, camera 320 may include a lens 321 that collects and focuses the light on to an image sensor 322. The image sensor 322 can be any of several off-the-shelf image complementary metal-oxide-semiconductor (CMOS) image sensor available such as the IMX104 by Sony Electronics. Optionally or additionally, one or more of camera 320 may be an infra-red camera or a camera at another wavelength or in some cases a multispectral camera in which case one or more of the image sensor 322 will be chosen for the appropriate wavelength(s) and/or combined with appropriate filters. The camera 320 may also comprise an image processor 323 that processes the image and compressed/encodes into a suitable format for transmission to display unit 350. The image processor 323 may also perform image processing functions such image segmentation, feature detection, and object recognition. It is anticipated that certain image processing will also be performed on the display unit 350 using CPU/GPU 351 and processing load-sharing between image processor 323 and CPU/GPU 351 will be optimized based of the needs of the particular application after considering performance factors such as power consumption and frame rate. A controller unit 324 may be a separate unit or integrated into processor 323 and performs the function of controlling the operation of camera 320 and receiving commands from CPU/GPU 351 in display unit 350 as well as sending messages to CPU/GPU 351.

In addition or alternatively, camera 320 may be one or more active depth cameras such as a Structured Light Camera, Time of flight (ToF) camera or a RGB-D camera using other suitable technologies. An RGB-D camera is an RGB camera that augments its image with depth information. In embodiments using such cameras, the image processor 323 may be configured to process 3D information (e.g. point clouds) in addition to 2D information. Examples of such cameras such as the Structure Sensor from Ocxipital of California, SWISS RANGER SR4000/4500 from MESA IMAGING of Zurich, Switzerland and CARMIN AND CAPRI series cameras from PRIMESENSE of Tel Aviv, Israel.

As shown in FIG. 2, camera 320 may also comprise interface 325 may include one or more components configured to transmit and receive data via a communication network, such as the Internet, a local area network, a workstation peer-to-peer network, a direct link network, a wireless network, or any other suitable communication platform. For example, interface 325 may include one or more modulators, demodulators, multiplexers, demultiplexers, network communication devices, wireless devices, antennas, modems, and any other type of device configured to enable data communication via a communication network. According to one embodiment, interface 325 may be coupled to or include wireless communication devices, such as a module or modules configured to transmit information wirelessly using Wi-Fi or Bluetooth wireless protocols.

As illustrated in FIG. 2, camera 320 may be powered by power supply 326, such as a battery, fuel cell, MEMs micro-generator, or any other suitable compact power supply. The camera 320 may also be powered by the display unit 350 using a wired connection.

It also anticipated that in certain embodiments of the camera 320, it can optionally comprise one or more inertial measurement units 120 as described herein. In such embodiments, several functional units such as power supply, processor, and interface units may be shared between camera 320 and inertial sensor.

The camera 320 in conjunction with display unit 350 forms a vision system capable of calculating and displaying the pose of fiducial markers 340 and relative pose between respective surgical instruments and/or implant rigidly coupled to it. For example, the camera 320 takes video images of one or more fiducial marker 340. Each image frame is analyzed and processed using algorithms that detect and localize specific visual features and/or patterns of the fiducial marker 340 such as pattern 180 in FIG. 3 or light emitting/reflecting light sources 150 in FIGS. 4-6. The algorithms analyze the 2D projection of the pattern or the light reflecting/emitting sources on the camera image plane and calculate the 3D pose of the fiducial marker 340 with respect to a reference coordinate system such as the camera. This final calculation relies in part on the calibration of the camera 320 which is performed prior to use. An example algorithm that performs the above sequence of operations in real-time is the open source AprilTag library (https://april.eecs.umich.edu/software/apriltag.html). It should be understood that AprilTag is only one example algorithm for processing images to detect and localize visual patterns of fiducial markers in order to calculate pose and that other algorithms may be used with the systems and methods described herein.

Although the vision system is capable of determining pose of the surgical instrument and/or implant on its own, system 300 is capable of fusing vision and inertial based methods to determine pose with greater resolution, speed, and robustness than is possible with systems that rely on any one type of information. For example, the pose information contained in the images, which is analyzed/processed as described above to obtain the pose in a reference coordinate system, can be fused with the pose information detected by the inertial measurement unit. In other words, the data streams from the inertial modalities (e.g., gyroscope, accelerometer, and/or magnetometer) may be “fused” with the pose obtained from the visual system using appropriate fusion and filtering techniques. An example of a technique that may be suitable for use with the systems and methods described herein is a Kalman Filter or an Extended Kalman Filter. The use of such fusion algorithms typically requires that coordinate frames of the vision system and inertial measurement unit be harmonized via a calibration procedure which can be performed prior to use.

It should be appreciated that the logical operations described herein with respect to the various figures may be implemented (1) as a sequence of computer implemented acts or program modules (i.e., software) running on a computing device (e.g., as included in the system of FIG. 2), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

An example method for estimating the relative pose between two or more implants is described herein. This disclosure contemplates that the method can be performed using the system 300 described with regard to FIGS. 1 and 2. The method can include establishing rigid coupling between a plurality of fiducial markers and a plurality of implants, respectively. As described above, a fiducial marker (e.g., one or more fiducial markers) such as fiducial marker 340 in FIGS. 1 and 2, for example, can be rigidly coupled to an implant (e.g., implant 333 or 334 in FIG. 1) using instrumentation such as a surgical instrument (e.g., surgical instrument 331 or 332 in FIG. 1). This disclosure contemplates that there can be more than one set of fiducial markers and implants, for example, the fiducial marker 340/rod 333 in FIG. 1 (first fiducial marker-implant set) and the fiducial marker 340/screw heads 334 in FIG. 1 (second fiducial marker-implant set). It should be understood that more than two fiducial marker-implant sets can be used. First information indicative of a respective relative pose between a respective fiducial marker and a respective implant for each fiducial marker-implant set can be estimated. As discussed above, this information can be estimated because of the rigid coupling between the fiducial marker 340 in FIG. 1 and the implant 333 (or implant 334) and the surgical instrument 331 (or surgical instrument 332). Additionally, second information indicative of a respective pose of each of the fiducial markers (e.g., fiducial markers 340 in FIGS. 1 and 2) can be received via an imaging device (e.g., camera 320 in FIGS. 1 and 2). As discussed above, the fiducial markers 340 can be tracked using the camera 320, and thus information indicative of the pose of the fiducial markers 340 can be obtained. Using the first information and the second information, a respective pose of each of the implants can then be estimated. Finally, a relative pose between at least two implants (e.g., both implants 333 and 334 in FIG. 1) can be estimated based on the respective pose of each of the at least two implants.

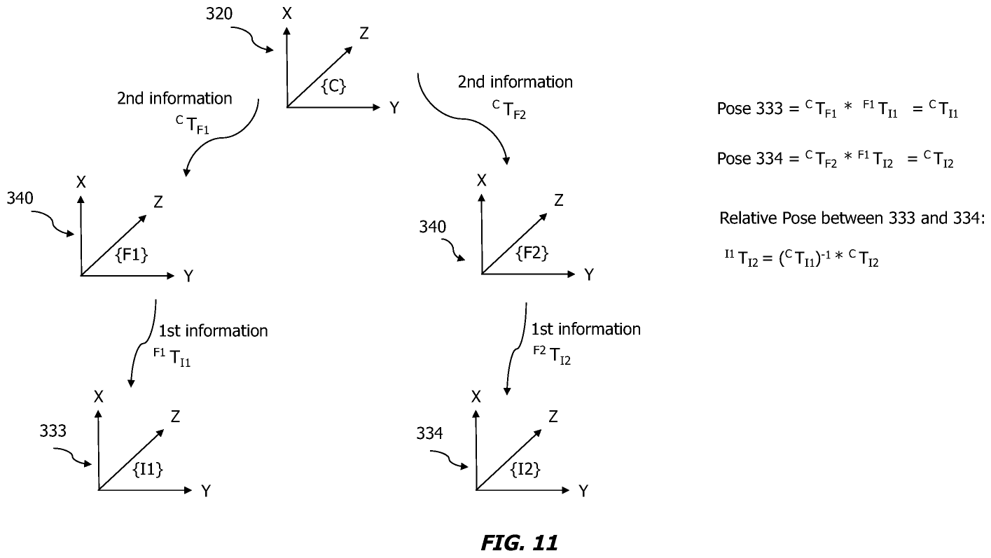

Referring to FIG. 11, example calculation of relative pose between at least two implants is shown. {C} represents the reference coordinate frame, in this case the coordinate frame of the imaging system or camera 320, {F1} and {F2} represent the respective coordinate frames of the fiducials markers 340 coupled to implants 333 and 334, respectively, and {I1} and {I2} represent the coordinate frames of the implants 333, 334, respectively. The relative pose of each of the implants relative to its respective fiducial marker, i.e. the first information, is represented by ^{F1}T_{I1} and ^{F2}T_{I2}. The pose of each of the fiducial markers, i.e. the second information, is represented by ^{C}T_{F1} and ^{C}T_{F2}. From the first and second information, the poses of both implants can be calculated as:

Pose of instrument 333=^{C}T_{F1}*^{F1}T_{I1}=^{C}T_{I1}

Pose of instrument 334=^{C}T_{F2}*^{F1}T_{I2}=^{C}T_{I2}

Relative pose between implants 333 and 334 can then be calculated as:

¹¹T_{I2}=(^{C}T_{I1})⁻¹*^{C}T_{I2}

The system can optionally register the anatomy and/or 3D imaging data like CT, MRI, etc. to allow for calculation of relative poses between implants or 3D visualization of the implants in anatomic reference frames. Techniques similar to those described for instrument registration herein may be utilized. One example process for anatomic registration is by attaching fiducial marker 340 and/or inertial measurement unit 120 to a calibrated elongate registration tool or pointer and either pointing or aligning the tool to certain bony landmarks that correspond to the same landmarks in an anatomical model. For example, system 300 may be configured to measure orientation of fiducial marker 340 or inertial measurement unit 120 while they are removably attached to an elongate registration tool that is aligned to specific pelvic, cervical, and/or lumbar landmarks. Alternatively, system 300 may be configured to measure the position of the tip of a pointer to which fiducial marker 340 is removable attached as the pointer palpates certain bony landmarks such as the spinous processes or collects points to map certain bony surfaces. Using pose associated between the anatomical landmarks and/or surfaces and pose of fiducial marker 340, a coordinate space that is representative of the anatomy can be derived.

Another example process for registration uses intraoperative images (such as fluoroscopic X-rays) taken at known planes (A-P or lateral), in some cases with identifiable reference markers on the anatomy. In such methods, one or more fiducial marker 340 or inertial measurement unit 120 may be rigidly attached to the imaging equipment or to a calibration fiducial visible in the image if pose information of the imaging equipment is required to achieve accurate registration.

In some implementations, the method can further include creating virtual models of the instruments or implants using pre-operative CAD data or intra-operative measurement and/or images. The pose information can be displayed by animating the virtual models.

In some implementations, the method can further include creating a virtual model of the surgical instrument.

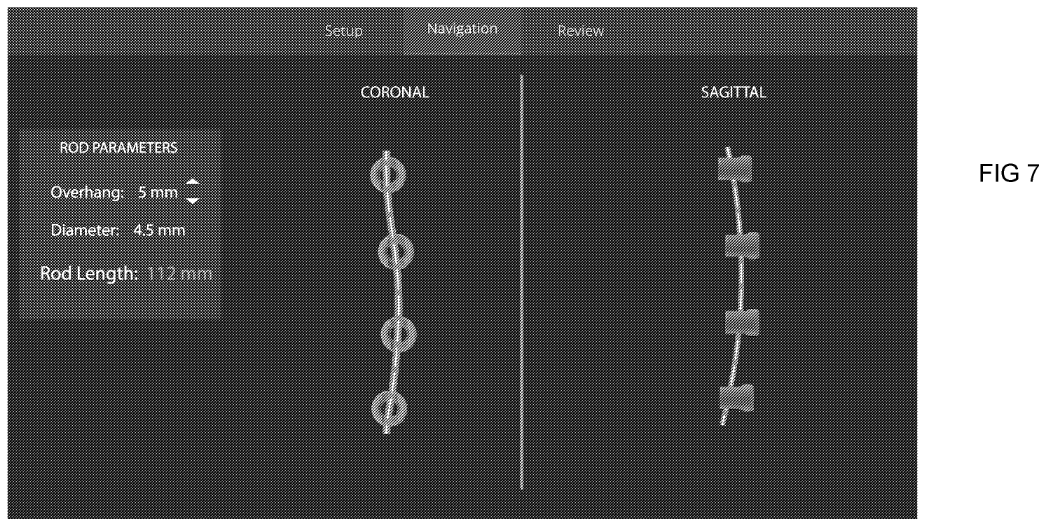

As shown in FIG. 7, the pose information of two or more implants can be utilized to update plan parameters for placement of one or more implants. Specifically, FIG. 7 shows plan parameters for a spinal rod implant that is passed through four screw heads. The plan parameters shown in FIG. 7 are rod parameters including overhang, diameter, and rod length. It should be understood that the plan parameters shown in FIG. 7 are only provided as examples and that other plan parameters can be used according to the implementations described herein. System 300 utilizes, at least in part, the measured pose information of the screw heads to update the plan parameters for the rod including rod contour/shape and size. Although the example in FIG. 7 is specific to rod contouring and sizing, the system 300 as described herein is capable of updating plan parameters for other implants based on measured pose parameters of two or more implants.

Augmented reality (AR) systems for use in a surgical environment are known in the art. For example, an AR system is described in WO2017/151752 to MIRUS LLC, published Sep. 8, 2017, entitled “AUGMENTED VISUALIZATION DURING SURGERY.” This disclosure contemplates acquisition and integration of multiple imaging modalities (magnetic resonance imaging (MRI), computed tomography (CT), fluoroscopy, etc.) by an AR system. Real time modification of the AR projection during surgery, for example as a patient's spine is manipulated, is contemplated. Additionally, the AR system can be integrated with surgical navigation for real time updates to a virtual model. The virtual model and AR projection can be used to see angles on the patient for lordosis, kyphosis, and sagittal alignment, for example. This disclosure contemplates use of algorithms making use of standing and lying pre-op and interop images to provide a standing projection based on correction being made.

It will be apparent to those skilled in the art that various modifications and variations can be made to the disclosed systems and methods for measuring orientation and position of an anatomy or surgical instrument in orthopedic arthroplastic procedures. Other embodiments of the present disclosure will be apparent to those skilled in the art from consideration of the specification and practice of the present disclosure. It is intended that the specification and examples be considered as exemplary only, with a true scope of the present disclosure being indicated by the following claims and their equivalents.

1. A method for estimating the relative pose between two or more implants, comprising:
establishing rigid coupling between a plurality of fiducial markers and a plurality of implants, respectively;
estimating first information indicative of a respective relative pose between a respective fiducial marker and a respective implant for each fiducial marker-implant set;
receiving, via an imaging device, second information indicative of a respective pose of each of the fiducial markers; and
estimating a respective pose of each of the implants based on the first information and the second information; and
estimating a relative pose between at least two implants based on the respective pose of each of the at least two implants.

2. The method of claim 1, wherein at least one of the implants is arranged under a patient's skin.

3. The method of claim 1, wherein at least one of the implants is not visible to the imaging device.

4. The method of claim 1, wherein estimating a respective pose of each of the implants based on the first information and the second information comprises, for each fiducial marker-implant set, extrapolating a pose of an implant from a pose of a fiducial marker to which the implant is rigidly coupled.

5. The method of claim 1, wherein establishing rigid coupling between a plurality of fiducial markers and a plurality of implants, respectively, comprises for each fiducial marker-implant set, rigidly attaching a fiducial marker and an implant at respective specific locations with respective specific relative orientations on a surgical instrument.

6. The method of claim 5, wherein the first information for each fiducial marker-implant set is estimated from: pre-operative information related to the dimensions of the surgical instrument and the implant; and a respective attachment point and a respective relative orientation of each of the fiducial marker and the implant on the surgical instrument.

7. The method of claim 1, wherein the first information is estimated preoperatively and/or intraoperatively via an instrument registration process.

8. The method of claim 7, wherein the instrument registration process comprises three-dimensional (3D) reconstruction of the implants from one or more images captured by the imaging device.

9. The method of claim 7, wherein the instrument registration process comprises using a point-pair registration technique.

10. The method of claim 7, wherein the instrument registration process comprises using a point-cloud registration technique.

11. The method of claim 1, further comprising receiving, via an inertial measurement unit, third information indicative of a respective pose of each of the implants.

12. The method of claim 11, further comprising fusing the second information and the third information, wherein the respective pose of each of the implants is estimated based on the first information and the fused second and third information.

13. The method of claim 12, wherein the second information and the third information are fused using a Kalman filter or an extended Kalman filter.

14. The method of claim 1, further comprising tracking the fiducial markers using the imaging device.

15. The method of claim 1, wherein at least one of the fiducial markers comprises a pattered or contoured surface.

16. The method of claim 1, wherein at least one of the fiducial markers comprises a light reflector or a light-emitting source.

17. The method of claim 1, wherein at least one of the fiducial markers comprises an inertial measurement unit.

18. The method of claim 17, wherein the inertial measurement unit comprises at least one of a gyroscope or an accelerometer.

19. The method of claim 1, further comprising displaying an estimated angle or a position between the at least two implants.

20. The method of claim 1, further comprising displaying an estimated angle or a position between at least one of the implants and an anatomic axis or plane.

21. The method of claim 1, further comprising:
creating a virtual model of at least one of the implants using pre-operative and/or intra-operative information; and
displaying a respective pose of the at least one of the implants by animating the virtual model.

22. The method of claim 21, further comprising displaying the animated virtual model using an augmented reality or virtual reality headset.

23. The method of claim 1, further comprising updating a plan parameter using the estimated respective pose of at least one of the implants.

24. The method of claim 23, wherein the plan parameter is an implant shape, an implant size, a trajectory, a surgical plan prediction, and/or a model of the at least one of the implants.

25. The method of claim 23, further comprising planning placement of the at least one of the implants.

26. The method of claim 23, further comprising displaying the plan parameter.

27. A system for estimating the relative pose between two or more implants, comprising:
an imaging device;
a plurality of fiducial markers coupled to a plurality of implants, respectively, using instrumentation; and
a processor communicatively coupled to the imaging device, the processor being configured to:
estimate first information indicative of a respective relative pose between a respective fiducial marker and a respective implant for each fiducial marker-implant set;
receive, via the imaging device, second information indicative of a respective pose of each of the fiducial markers;
estimate a respective pose of each of the implants based on the first information and the second information; and
estimate a relative pose between at least two implants based on the respective pose of each of the at least two implants.

28. The system of claim 27, wherein the fiducial markers are rigidly coupled to the implants using instrumentation.

29. The system of claim 28, wherein the instrumentation comprises at least one surgical instrument.

30. The system of claim 29, wherein a fiducial marker and an implant of each fiducial marker-implant set is rigidly attached at respective specific locations with respective specific relative orientations on a surgical instrument.

31. The system of claim 30, wherein the first information for each fiducial marker-implant set is estimated from: pre-operative information related to the dimensions of the surgical instrument and the implant; and a respective attachment point and a respective relative orientation of each of the fiducial marker and the implant on the surgical instrument.

32. The system of claim 27, wherein the first information is estimated preoperatively or intraoperatively via an instrument registration process.

33. The system of claim 32, wherein the instrument registration process comprises three-dimensional (3D) reconstruction of the implants from one or more images captured by the imaging device.

34. The system of claim 32, wherein the instrument registration process comprises using a point-pair registration technique.

35. The system of claim 32, wherein the instrument registration process comprises using a point-cloud registration technique.

36. The system of claim 27, wherein estimating a respective pose of each of the implants based on the first information and the second information comprises, for each fiducial marker-implant set, extrapolating a pose of an implant from a pose of a fiducial marker to which the implant is rigidly coupled.

37. The system of claim 27, wherein at least one of the fiducial markers comprises an inertial measurement unit configured to provide third information indicative of a respective pose of at least one of the implants.

38. The system of claim 37, wherein the processor is further configured to fuse the second information and the third information, wherein the respective pose of the at least one of the implants is estimated based on the first information and the fused second and third information.

39. The system of claim 38, wherein the second information and the third information are fused using a Kalman filter or an extended Kalman filter.

40. The system of claim 27, wherein the imaging device is mounted on a surgical table, cart, pole, augmented reality headset, or virtual reality headset.

41. The system of claim 27, wherein the imaging device is integrated with a surgical light.

42. The system of claim 27, wherein the processor is further configured to update a plan parameter using the estimated respective pose of at least one of the implants.

43. The system of claim 42, wherein the plan parameter is an implant shape, an implant size, a trajectory, a surgical plan prediction, and/or a model of the at least one of the implants.

44. The system of claim 42, wherein the processor is further configured to plan placement of the at least one of the implants.

45. The system of claim 42, wherein the processor is further configured to display the plan parameter on a display device.
